(19) United States
(12) Plant Patent Application Publication
(10) Pub. No.: US 2011/0162119 P1
(43) Pub. Date: Jun. 30, 2011

(54) Strawberry plant named Sweet Ann

(50) ;

(75) Inventor: Jimmy Bagdasarian, Santa Cruz, CA (US)

(73) Assignees: Mr.Jimmy Haig Bagdasarian, Santa Cruz, CA (US); Lassen Canyon Nursery, Inc, Redding, CA (US)

(21) Appl. No.: 12/655,174

(22) Filed: Dec. 24, 2009

(51) Int. Cl. A01H 5/00

(52) U.S. Cl. PLT/208

(57) ABSTRACT

A new and distinct variety of strawberry (Fragaria×ananassa) which originated from seed produced by a hand-pollinated cross between two Lassen Canyon Nursery proprietary cultivars designated “4A28” and “10B131”. The new variety is distinguished by its open plant architecture, its high vigor which results in high yields of very large to large fruit of exceptional flavor when grown under the ambient condition of coastal California.

### BOTANICAL/COMMERCIAL CLASSIFICATION

Fragaria×ananassa/Strawberry plant

### VARIETAL DENOMINATION

cv ‘Sweet Ann’

### REFERENCES CITED

U.S. patent documents
U.S. Plant Pat. No. 12,403 February 2002 Small et al.
U.S. Plant Pat. No. 9,320 October 1995 Bagdasarian et al.
U.S. Plant Pat. No. 16,228 January 2006 Shaw et al.

### SUMMARY OF THE INVENTION

### Background of the New Variety

The present invention relates to a new and distinct variety of strawberry (Fragaria×ananassa) plant which is named “Sweet Ann” and more particularly to a strawberry plant that is distinguished by its production of very large to large fruit of exceptional flavor, medium skin firmness and medium red to full red external color. Plants were asexually propagated in Shastina, Calif. and the plants evaluated and selected in Oxnard, Calif. Contrast is made to ‘Albion’ (U.S. Pat. No. 16,228) and Aromas (U.S. Plant Pat. No. 10,451) and other commercially grown varieties as indicated in the tables below. The high productivity and, exceptional flavor make the new variety highly competitive in the strawberry industry.

### Origin of the Variety

This new and distinct day-neutral strawberry cultivar was the product of a controlled cross carried out at Santa Cruz, Calif., USA. Sweet Ann was selected from a cross between two proprietary plants designated ‘4A28’ a female, and 10B131 a male from the Lassen Canyon Nursery/Bagdasarian breeding program. The seeds resulting from this cross were germinated in a greenhouse at Redding, Calif., USA. The resulting seedlings were transplanted to an open field in Shastina, Calif. and allowed to produce daughter plants by asexual propagation (i.e. by runners). The seedlings were harvested and transplanted to Oxnard, Calif., where they were regularly observed in a breeding program plot and subjected to detailed evaluation. ‘Sweet Ann was selected from among various sibling genotypes as the 29^{th} selection in 2007 and thus designated 16F29. It has been asexually propagated by runners, annually, and further test plantings have established that the vegetative and fruit characteristics of the propagules are identical to the initial daughter plants.

### Summary of the Variety

During the 2007 season Sweet Ann, then known as (16-f-29), was chosen primarily on the basis of its productivity, fruit flavor, and appearance. The cultivar was further advanced through plot selection trials during the 2008 and 2009 seasons. When grown in the marine moderated conditions in three different regions of coastal California, ‘Sweet Ann’ distinguishes itself by a combination of three characteristics: (a) high productivity (averaging 2612 crates per acre of marketable fruit per acre in a 20-week commercial trial in Irvine, Calif.; (b) very large to large, conically shaped fruit (averaging >33 grams per berry) and a vigorous plant that maintained its open architecture.) See Table 4.

Independent test plot work was done in Irvine, Oxnard, Santa Maria, and Watsonville, Calif. In trials at Irvine, Oxnard, Santa Maria, and Watsonville, Calif., evaluation of the cultivar included yield, yield distribution, fruit size, fruit shape, external and internal fruit color, fruit resistance to bruising and abrasion (also known as “shipability”), fruit shelf life, fruit flavor, overall appearance, tolerance to disease and rain damage, plant architecture, ease of harvest, truss type and propensity for runner production. During the testing period Sweet Ann demonstrated the desirable combinations of traits disclosed herein.

The new cultivar has been meristemed and planting stock from meristemed plants are growing in a screenhouse located in Redding, Calif. It has also been asexually reproduced by use of runner production in Siskiyou, Shasta, and San Joaquin counties, Calif. The characteristic traits associated with the Sweet Ann variety are fixed and remain true to type through successive generations of asexual reproduction. Further, three plants of the Sweet Ann variety were submitted for allelic fingerprint analysis to Dr. Parm Randhawa at California Seed & Plant Lab., Inc. 7877 Pleasant Grove Road, Elverta, Calif. Dr. Randhawa found the Sweet Ann variety to be unique as compared to all other varieties in their database. (See FIG. 7.)

### Summary of the Variety

The new strawberry variety exhibits the following combinations of characteristics:
(a) The variety is a day-neutral cultivar,
(b) The variety produces large sized, attractive, generally well shaped long conical fruit that has a good acid-sugar balance and is sweet tasting.
(c) The fruit has a glossy medium red exterior and medium red interior.
(d) The variety produces few runners in the fruiting field.

The new cultivar possesses characteristics that are commonly sought by commercial strawberry growers. Sweet Ann produced moderately firm berries that are large size, firm and have an excellent flavor. The BRIX levels of Sweet Ann are compared with Albion and San Andreas. Table 3. The sugar levels were measured with a refractometer manufactured by Spectrum Technologies, Inc. 12360 S. Industrial Dr. East, Plainfield, Ill. 60544. The berries retain their firmness, color and desert quality even following long distance shipment.

The leaf and fruit color of the new variety is readily distinguished from that of other commercially grown strawberry varieties. Table 1 compares these characteristics of the ‘Sweet Ann’ with the ‘Chandler’, ‘Camarosa’, ‘Albion’, and ‘Catalina’ as shown in Table 1. Table 2 compares the mid-tier leaf width, mid-tier leaf length, petole length and plant height of the ‘Sweet Ann’ with that of ‘Albion’ and ‘Aromoas’. Color terminology used herein is in accordance with Pantone Color Formula Guide GP 1201, Pantone, Inc. World Headquarters 590 Commerce Boulevard, Carlstadt, N.J. 07072-3098 USA.

The new cultivar has been meristemed and is growing in a screenhouse located in Redding, Calif. It has also been asexually reproduced by use of runner production in Siskiyou, Shasta, and San Joaquin counties, Calif.

The new plant of the present invention has been named ‘Sweet Ann’.

### DETAILED DESCRIPTION

The described plants have been asexually reproduced by the use of runners, and were planted outdoors in late April in Macdoel, Calif. The ‘Sweet Ann’ variety has not been observed under all possible environmental conditions and thus the variety may vary in detail depending upon factors including, but not limited to weather, day length, soil type and geographical location.
Botanical class: FRAGARIA×ANANASSA CV. ‘Sweet Ann’
Plant:
   Type.—Day Neutral.
   Configuration.—Globose and open plant density.
   Vigor.—Strong.
Foliage:
   Size.—The terminal leaflets are greater in length than width and display an average 67 mm in width and approximately 83 mm in length. The petiole length averages 220 mm in length and plant height averages 380 mm.
   Margin.—Commonly crenate.
   Shape.—Orbicular.
   Base.—Obtuse.
   Cross-section.—Moderately concave.
   Blistering.—Absent or very weak.
   Glossiness.—Medium.
   Color.—Adaxial Surface: green 364U — Abaxial Surface: green 370U.
   Petiole texture.—Medium pubescence with hairs directed outwards.
   Petiole color.—Green 383U.
   Stipules.—Commonly anthocyanin coloration is absent or very weak.
   Stolons.—Weak presence.
Inflorescence:
   Flowering time.—Early.
   Position.—Approximately at or above canopy height.
   Size.—Long and averages in mid-season plants at 33 cm.
   Petals.—Average 5.4 petals that are overlapping.
   Calyx.—Color adaxial 364U and abaxial 370U that are larger in size relative to the corolla.
Fruit:
   Bearing.—Remontant.
   Shape.—Generally conical and occasional wedge-shaped and commonly the length is greater than the width.
   Length.—Approximately 49.4 mm on average.
   Achenes.—Approximately level with the fruit surface.
   Glossiness.—Strong.
   External color.—Red 364U.
   Internal color.—Red 1788C.
   Fruit center.—Commonly solid with some hollowness.

### BRIEF DESCRIPTION OF THE FIGURES

The accompanying color photographs show, as nearly true as it is reasonably possible to make the same in color illustrations of this character, typical specimens of the new cultivar designated ‘Sweet Ann’, also known as ‘16F29’, including fruit, foliage and flower, as follows:

FIG. 1 shows typical fruiting and field characteristics. (Photos all taken August 2009.)

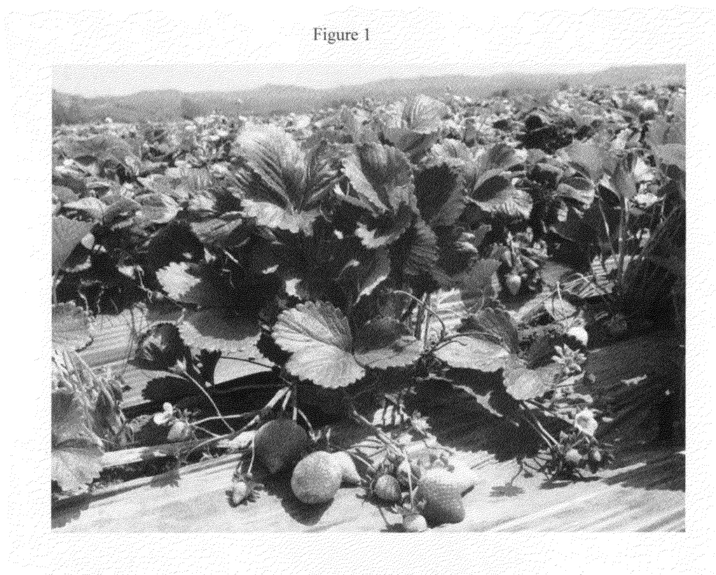

FIG. 2 shows typical leaf structure.

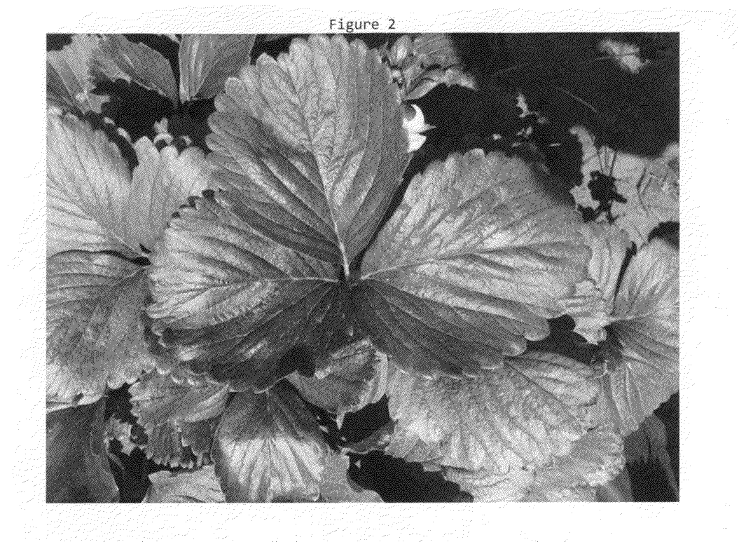

FIG. 3 shows typical mid-season fruit.

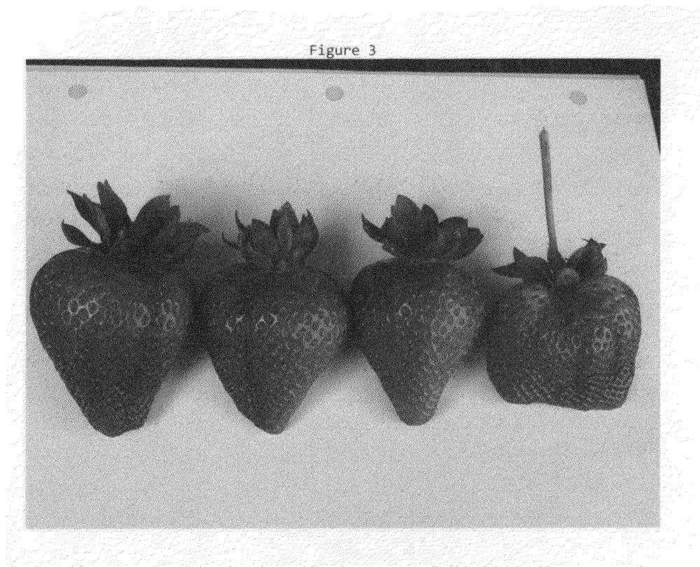

FIG. 4 shows typical mid-season calyx.

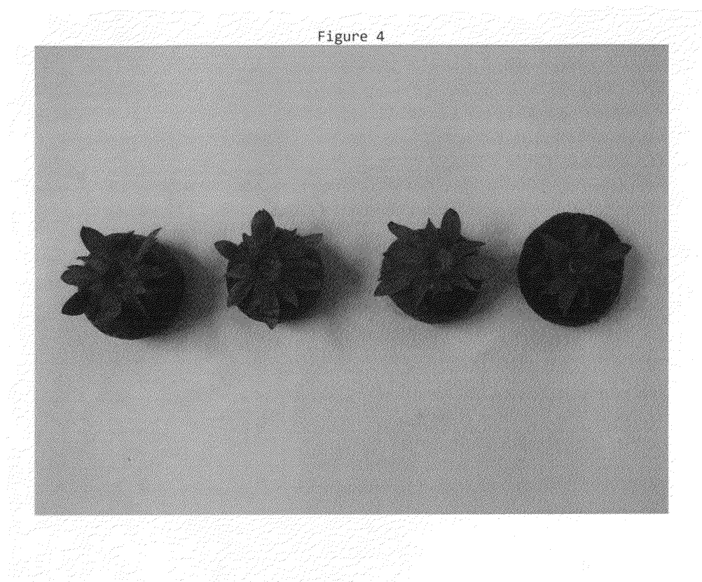

FIG. 5 shows typical mid-season inflorescence.

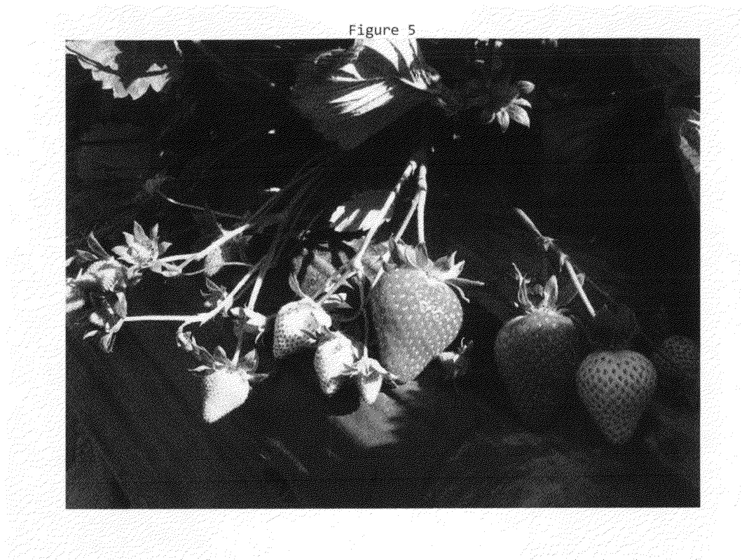

FIG. 6 shows typical mid-season fruit interiors.

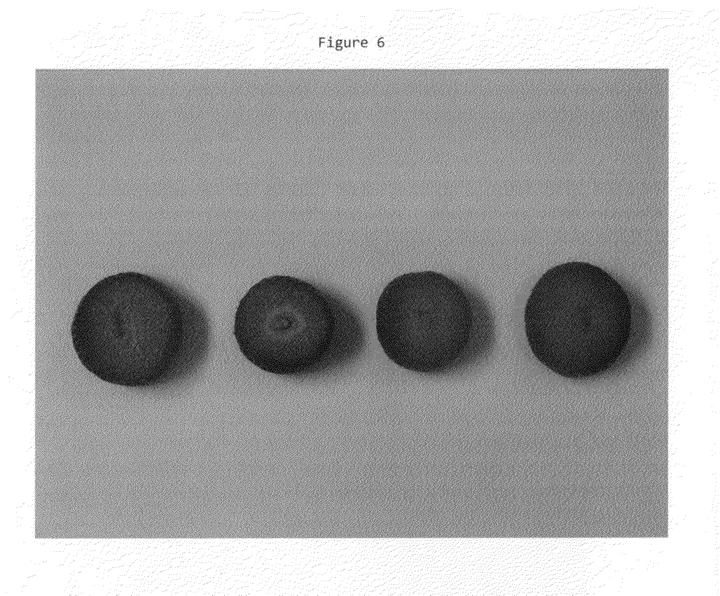

FIG. 7. Shows the allelic fingerprint.

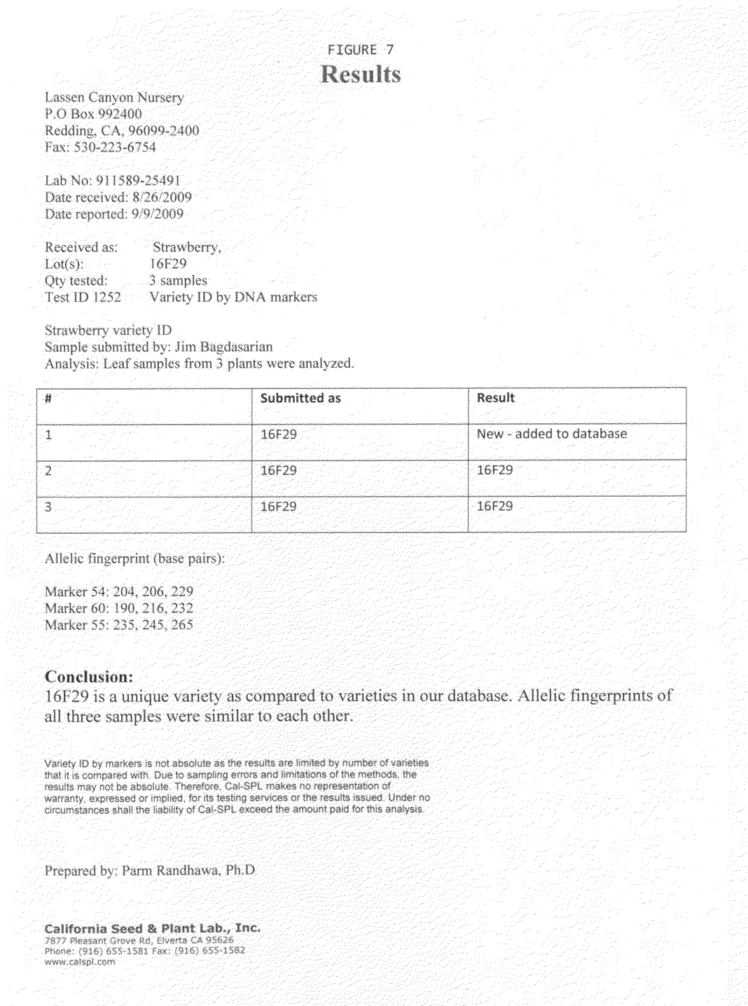

The following Table 1 provides information regarding visual comparisons of Sweet Ann leaf and fruit color to leaf and fruit color of ‘Chandler’, ‘Camarosa,’ ‘Albion,’ and ‘Catalina.’

TABLE 1
visual comparisons of ‘Sweet Ann’ leaf color to ‘Albion’ and leaf and
fruit color to ‘Chandler’, ‘Camarosa’, ‘Albion’ and ‘Catalina’.

| | Adaxial | Abaxial | External | Internal |
|---|---|---|---|---|
| | Leaf | leaf | Fruit | Fruit |
| Sweet Ann’ | 364U | 370U | 185C | 1788C |
| ‘Chandler’ | 343C | 339U | 186C | 179C |
| ‘Camarosa’ | 349C | 348U | 193C | 185C |
| ‘Albion’ | 357U | 7490U | n/a | n/a |
| ‘Catalina’ | 343C | 349U | 193C | 185C |

The following Table 2 provides foliar characteristics for ‘Sweet Ann’, ‘Albion’ and ‘Aromas’:

TABLE 2
Foliar characteristics for ‘Sweet Ann’, ‘Albion’ and ‘Aromas’

| | CULTIVAR | | |
|---|---|---|---|
| Foliar Character | Sweet Ann’ | ‘Albion’ | ‘Aromas’ |
| Mid-tier leaf | | | |
| Width | | | |
| Mean | 67 | 73 | 92 |
| Range | 52-88 | 50-95 | 67-100 |
| Mid-tier leaf | | | |
| Length | | | |
| Mean | 83 | 68 | 74 |
| Range | 70-104 | 50-95 | 65-85 |
| Petiole | | | |
| Length | | | |
| Mean | 220 | 68 | 156 |
| Range | 150-280 | 50-95 | 135-200 |
| plant | | | |
| Height | | | |
| Mean | 380 | 252 | 257 |
| Range | 290-480 | 210-270 | 230-330 |

TABLE 3
BRIX Levels for Sweet Ann, Albion, and San Andreas
(Data Taken on Nov. 11, 2009)

| | | | | | |
|---|---|---|---|---|---|
| Sweet Ann | 13 | 12.1 | 11.9 | 10.5 | 13.2 |
| Albion | 11 | 11 | 11.2 | 11.6 | No data |
| San Andreas | 7 | 10 | 5.75 | 8.2 | No data |

TABLE 4
Production Comparison between Sweet Ann and Ventana,
Camarosa, Albion, and Palomar from Dec. 12, 2008
through Mar. 2, 2009 in Irvine, California

| Variety | Ave. Grams per Berry | Ave. Crates per Acre |
|---|---|---|
| Sweet Ann | 33 | 2617 |
| Ventana | 36 | 1741 |
| Camarosa | 29.4 | 1984 |
| Albion | 28 | 1803 |
| Palomar | 36.3 | 1895 |

We claim:

1. A new and distinct strawberry plant as illustrated and described, characterized by an open plant architecture, very large to large fruit with exceptional flavor and desert quality when grown in coastal regions of California.
